(19) United States
(12) Patent Application Publication
(10) Pub. No.: US 2002/0022864 A1
(43) Pub. Date: Feb. 21, 2002

(54) Multipolar electrode system for radiofrequency ablation

(76) Inventors: David M. Mahvi, Middleton, WI (US); John G. Webster, Madison, WI (US); Fred T. Lee JR., Madison, WI (US); Stephen T. Staelin, Madison, WI (US); Dieter Haemmerich, Madison, WI (US); Supan Tungjitkusolmun, Bangkok (TH)

Correspondence Address: QUARLES & BRADY LLP, 411 E. WISCONSIN AVENUE, SUITE 2040, MILWAUKEE, WI, 53202-4497, US

(21) Appl. No.: 09/873,541

(22) Filed: Jun. 4, 2001

Related U.S. Application Data
(60) Provisional application No. 60/210,103, filed on Jun. 7, 2000.

(51) Int. Cl. A61N 1/00

(52) U.S. Cl. 607/2

(57) ABSTRACT

In radio frequency ablation, larger lesion volumes are obtained for a given energy delivery by energizing at least two electrodes on either side of the tumor so that current is focused between them rather than dispersed radially to a large area ground plate. Modified standard umbrella probes may be used or a specialized dual electrode array may be fabricated for simplified use. Differential impedance between tumor and non-tumor tissues at certain frequencies is exploited to further improve lesion shape and size.

### CROSS-REFERENCE TO RELATED APPLICATIONS

This application claims the benefit of provisional application Serial No. 60/210,103 filed Jun. 7, 2000 entitled Multipolar Electrode System for Radiofrequency Ablation.

### STATEMENT REGARDING FEDERALLY SPONSORED RESEARCH OR DEVELOPMENT

### --

### BACKGROUND OF THE INVENTION

The present invention relates to electrodes for radiofrequency ablation of tumors and the like, and in particular to a multipolar electrode system suitable for the ablation of liver tumors.

Ablation of tumors, such as liver (hepatic) tumors, uses heat or cold to kill tumor cells. In cryosurgical ablation, a probe is inserted during an open laparotomy and the tumor is frozen. In radiofrequency ablation (RFA), an electrode is inserted into the tumor and current passing from the electrode into the patient (to an electrical return typically being a large area plate on the patient's skin) destroys the tumor cells through resistive heating.

A simple RFA electrode is a conductive needle having an uninsulated tip placed within the tumor. The needle is energized with respect to a large area contact plate on the patient's skin by an oscillating electrical signal of approximately 460 kHz. Current flowing radially from the tip of the needle produces a spherical or ellipsoidal zone of heating (depending on the length of the exposed needle tip) and ultimately a lesion within a portion of the zone having sufficient temperature to kill the tumor cells. The size of the lesion is limited by fall-off in current density away from the electrode (causing reduced resistive heating), loss of heat to the surrounding tissue, and limits on the amount of energy transferred to the tissue from the electrode. The electrode energy is limited to avoid charring, boiling and vaporization the tissue next to the electrode, a condition that greatly increases the resistance between the electrode and the remainder of the tumor. The tissue next to the electrode chars first because of the high current densities close to the electrode and thus creates a bottleneck in energy transfer.

Several approaches have been developed to increase energy delivered to tissue without causing charring. A first method places temperature sensors in the tip of the electrode to allow more accurate monitoring of temperatures near the electrode and thereby to allow a closer approach to those energies just short of charring. A second method actively cools the tip of the electrode with circulated coolant fluids within the electrode itself. A third method increases the area of the electrode using an umbrella-style electrode in which three or more electrode wires extend radially from the tip of the electrode shaft, after it has been positioned in the tumor. The greater surface area of the electrode reduces maximum current densities. The effect of all of these methods is to increase the amount of energy deposited into the tumor and thus to increase the lesion size allowing more reliable ablation of more extensive tumors.

A major advantage of RFA in comparison to cryosurgical ablation is that it may be delivered percutaneously, without an incision, and thus with less trauma to the patient. In some cases, RFA is the only treatment the patient can withstand. Further, RFA can be completed while the patient is undergoing a CAT scan.

Nevertheless, despite the improvements described above, RFA often fails to kill all of the tumor cells and, as a result, tumor recurrence rates of as high as 40% have been reported.

### SUMMARY OF THE INVENTION

The present inventors have modeled the heating zone of standard RFA electrodes and believe that the high recurrence rate currently associated with RFA may result in part from limitations in the lesion size and irregularities in the lesion shape that can be obtained with these electrodes. Current lesion sizes may be insufficient to encompass the entire volume of a typical hepatic tumor particularly in the presence of nearby blood vessels that act as heat sinks, carrying away energy to reduce the lesion size in their vicinity.

In order to overcome the energy limitations of current electrode designs, the present inventors have adopted a multipolar electrode design that increase lesion size by “focusing” existing energy on the tumor volume between two or more electrodes. By using axially displaced umbrella electrodes supported by outwardly non-conductive shafts, a more regular lesion area is created than is provided by a single umbrella electrode and the lesion produced is greater in volume than would be obtained by a comparable number of monopolar umbrella electrodes operating individually.

Specifically, the present invention provides a method of tumor ablation in a patient including the steps of inserting a first electrode percutaneously at a tumor volume, the first electrode having a first support shaft with a first shaft tip, so that the first shaft tip is at first locations adjacent to the tumor volume and offset from a center of the tumor volume and inserting a second electrode percutaneously at the tumor volume, the second electrode having a second support shaft with a second shaft tip, so that the second support shaft is generally parallel and adjacent to the first support shaft, and so that the second shaft tip is at a second location opposed and at a predetermined separation from the first location about the tumor volume. First and second electrically isolated wire umbrella electrodes sets are extended radially from the first and second shaft tips to an extension radius; and a power supply is connected between the first and second electrode sets to induce a current flow between them through the tumor volume whereby current induced heating is concentrated in the tumor volume.

It is thus one object of the invention to provide a better shaped and substantially increased lesion volume while working within the energy limits imposed by local tissue boiling, vaporization and charring. The use of multiple radially displaced umbrella electrode sets communicating current between them delivers more energy to the tumor without necessarily increasing the total amount of energy delivered. The voltage may be an oscillating voltage waveform having substantial energy in the spectrum below 500 kHz and preferably below 100 kHz.

The present inventors have further recognized that the impedance of tumor tissue differs significantly from that of regular tissue at frequencies below 100 kHz and especially below 10 kHz. Thus is another object of the invention to exploit this discovery to preferentially ablate tumor tissue by proper selection of the frequency of the electrical energy.

The method may include the steps of monitoring the temperature at the first or second electrode and controlling the voltage delivered to the electrodes as a function of that temperature.

Thus it is another object of the invention to employ temperature feedback systems of the prior art with the present invention to increase, to the extent possible, the total energy delivered by the electrodes.

The first and second electrodes may be umbrella electrodes having at least two electrode wires extending radially from a support shaft to a radius from the support shaft and the first and second locations may be separated by an amount less than six times (and preferably four times) the maximum radius to which the electrode wires are extended.

Thus it is another object of the invention to separate the electrodes by an amount that maximizes the useful size of the contiguous lesion volume.

The method may include the further step of placing an additional conductor in contact to provide a diffuse return path for current (for example), a conductive plate against the skin of the patient.

Thus it is another object of the invention to provide even greater control over the current flow through the tumor, particularly in situations where inhomogeneities in the tissue would normally render one electrode much cooler than the other. Such inhomogeneities can include, for example, nearby blood vessels which carry heat away from nearby tissue. By using the conductive plate to augment current flow in one electrode, energy delivery at that electrode may be increased without changing the energy delivery at the other electrode.

The method may include the steps of placing at least one third electrode percutaneously at a third location different from the first and second locations but adjacent to the tumor and offset from the center of the tumor volume and monitoring the temperature at the first, second and third electrodes.

Thus it is another object of the invention to apply the present principles of this invention to multi-electrode systems that may define arbitrary volumes and accurately control temperature within those volumes for complete tumor ablation.

A support shaft having a shaft tip and a shank portion having a predetermined separation from the shaft tip, and sized for percutaneous placement of the shaft tip adjacent to the first location and the shank portion adjacent to the second location may have first and second wire electrode sets extensible radially from the support shaft at the first and second locations. A power supply may be connected between the first and second electrode sets to induce a current flow there between.

Thus it is another object of the invention to provide a single apparatus for practicing the above method. A single shaft supporting the first and second wire sets in a predetermined separation corresponding to particular tissue characteristics and tumor sizes, simplifies use of the method. Multiple different shafts with different separations can be provided for different tumor sizes.

The ends of the electrode wire sets removed from the support shaft may be insulated.

It is thus another object of the invention to eliminate hot spots caused by high current densities at the tips of electrodes even in umbrella-type electrodes.

The invention further insulating cover may extend between the shaft and the shaft tip.

Thus it is another object of the invention to prevent short circuit paths between the electrode sets through tissue and to the shaft.

The foregoing and other objects and advantages of the invention will appear from the following description. In this description, reference is made to the accompanying drawings, which form a part hereof, and in which there is shown by way of illustration, a preferred embodiment of the invention. Such embodiment and its particular objects and advantages do not define the scope of the invention, however, and reference must be made therefore to the claims for interpreting the scope of the invention.

### BRIEF DESCRIPTION OF THE DRAWINGS

FIG. 1 is a perspective view of two umbrella electrode assemblies providing first and second electrode wires deployed per the present invention at opposite edges of a tumor to create a lesion encompassing the tumor by a passing current between the electrodes;

FIG. 2 is a schematic representation of the electrodes of FIG. 1 as connected to a voltage controlled oscillator and showing temperature sensors on the electrode wires for feedback control of oscillator voltage;

FIG. 3 is a fragmentary cross-sectional view of a tip of a combined electrode assembly providing for the first and second electrode wires of FIG. 1 extending from a unitary shaft arranging the wires of the first and second electrodes in concentric tubes and showing an insulation of the entire outer surface of the tubes and of the tips of the electrode wires;

FIG. 4 is a simplified elevational cross-section of a tumor showing the first and second electrode positions and comparing the lesion volume obtained from two electrodes operating per the present invention, compared to the lesion volume obtained from two electrodes operating in a monopolar fashion;

FIG. 5 is a figure similar to that of FIG. 2 showing electrical connection of the electrodes of FIG. 1 or FIG. 3 to effect a more complex control strategy employing temperature sensing from each of the first and second electrodes and showing the use of a third skin contact plate held in voltage between the two electrodes so as to provide independent current control for each of the two electrodes;

FIG. 6 is a graph plotting resistivity in ohms-centimeters vs. frequency in Hz for tumorous and normal liver tissue, showing their separation in resistivity for frequencies below approximately 100 kHz;

FIG. 7 is a figure similar to that of FIGS. 2 and 5 showing yet another embodiment in which wires of each of the first and second electrodes are electrically isolated so that independent voltages or currents or phases of either can be applied to each wire to precisely tailor the current flow between that wire and the other electrodes; and

FIG. 8 is a flow chart of a program as may be executed by the controller of FIG. 7 in utilizing its multi-electrode control.

### DETAILED DESCRIPTION OF THE INVENTION

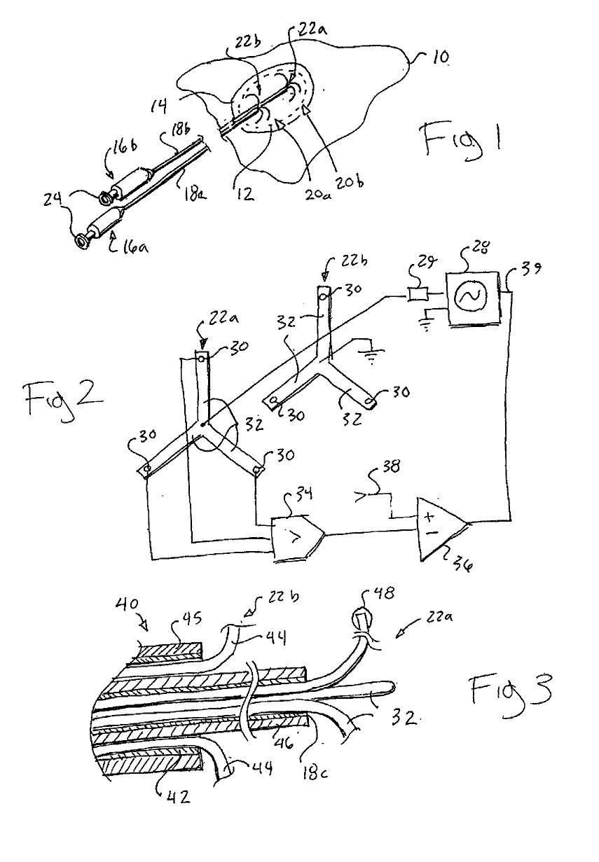

Referring now to FIG. 1, a liver 10 may include a tumor 12 about which a lesion 14 will be created by the present invention using two umbrella-type electrode assemblies 16a and 16b having a slight modification as will be disclosed below. Each electrode assembly 16a and 16b has a thin tubular metallic shaft 18a and 18b sized to be inserted percutaneously into the liver 10. The shafts 18a and 18b terminate, respectively, at shaft tips 20a and 20b from which project trifurcated electrodes 22a and 22b are formed of wires 32. The wires 32 are extended by means of a plunger 24 remaining outside the body once the shafts 18a and 18b are properly located within the liver 10 and when extended, project by an extension radius separated by substantially equal angles around the shaft tips 20a and 20b. The exposed ends of the wires 32 are preformed into arcuate form so that when they are extended from the shafts 18a and 18b they naturally splay outward in a radial fashion.

Umbrella electrode assemblies 16a and 16b of this type are well known in the art, but may be modified, in one embodiment of the invention, by providing electrical insulation to all outer surfaces of the shafts 18a and 18b, in contrast to prior art umbrella electrode assemblies which leave the shaft tips 20a and 20b uninsulated, and by insulating the tips of the exposed portions of the wires 32. The purpose and effect of these modifications will be described further below.

Per the present invention, the first electrode 22a is positioned at one edge of the tumor 12 and the other electrode 22b positioned opposite the first electrode 22a across the tumor 12 center. The term “edge” as used herein refers generally to locations near the periphery of the tumor 12 and is not intended to be limited to positions either in or out of the tumor 12, whose boundaries in practice, may be irregular and not well known. Of significance to the invention is that a part of the tumor 12 is contained between the electrodes 22a and 22b.

Referring now to FIGS. 1 and 2, electrode 22a may be attached to a voltage controlled power oscillator 28 of a type well known in the art providing a settable frequency of alternating current power whose voltage amplitude (or current output) is controlled by an external signal. The return of the power oscillator 28 is connected to electrodes 22b also designated as a ground reference. When energized, power oscillator 28 induces a voltage between electrodes 22a and 22b causing current flow therebetween.

Referring now to FIG. 4, prior art operation of each electrode 22a and 22b being referenced to a skin contract plate (not shown) would be expected to produce lesions 14a and 14b, respectively, per the prior art. By connecting the electrodes as shown in FIG. 2, however, with current flow therebetween, a substantially larger lesion 14c is created. Lesion 14c also has improved symmetry along the axis of separation of the electrodes 22a and 22b. Generally, it has been found preferable that the electrodes 22a and 22b are separated by 2.5 to 3 cm for typical umbrella electrodes or by less than four times their extension radius.

Referring again to FIG. 2, temperature sensors 30, such as thermocouples, resistive or solid-state-type detectors, may be positioned at the distal ends of each of the exposed wires 32 of the tripartite electrodes 22a and 22b. For this purpose, the wires 32 may be small tubes holding small conductors and the temperature sensors 30 as described above. Commercially available umbrella-type electrode assemblies 16a and 16b currently include such sensors and wires connecting each sensor to a connector (not shown) in the plunger 24.

In a first embodiment, the temperature sensors 30 in electrode 22a are connected to a maximum determining circuit 34 selecting for output that signal, of the three temperature sensors 30 of electrode 22, that has the maximum value. The maximum determining circuit 34 may be discrete circuitry, such as may provide precision rectifiers joined to pass only the largest signal, or may be implemented in software by first converting the signals from the temperature sensors 30 to digital values and determining the maximum by means of an executed program on a microcontroller or the like.

The maximum value of temperature from the temperature sensors 30 is passed by a comparator 36 (which also may be implemented in discrete circuitry or in software) which compares the maximum temperature to a predetermined desired temperature signal 38 such as may come from a potentiometer or the like. The desired temperature signal is typically set just below the point at which tissue boiling, vaporization or charring will occur.

The output from the comparator 36 may be amplified and filtered according to well known control techniques to provide an amplitude input 39 to the power oscillator 28. Thus it will be understood that the current between 22a and 22b will be limited to a point where the temperature at any one temperature sensors 30 approaches the predetermined desired temperature signal 38.

While the power oscillator 28 as described provides voltage amplitude control, it will be understood that current amplitude control may instead also be used. Accordingly, henceforth the terms voltage and current control as used herein should be considered interchangeable, being related by the impedance of the tissue between the electrodes 22b and 22a.

In an alternative embodiment, current flowing between the electrodes 22a and 22b, measured as it flows from the power oscillator 28 through a current sensor 29, may be used as part of the feedback loop to limit current from the power oscillator 28 with or without the temperature control described above.

In yet a further embodiment, not shown, the temperature sensors 30 of electrode 22b may also be provided to the maximum determining circuit 34 for more complete temperature monitoring. Other control methodologies may also be adopted including those provided for weighted averages of temperature readings or those anticipating temperature readings based on their trends according to techniques known to those of ordinary skill in the art.

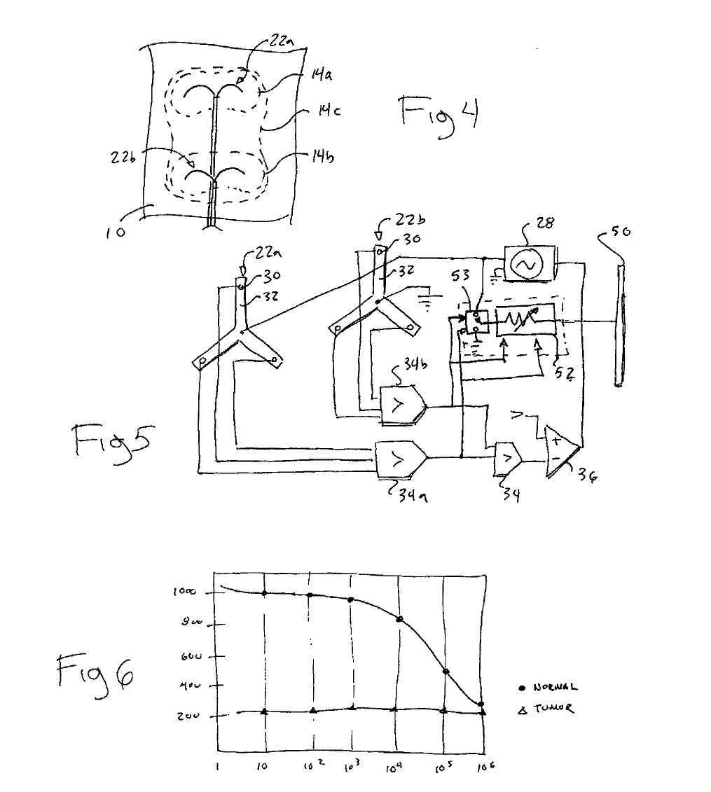

Referring now to FIG. 3, the difficulty of positioning two separate electrode assemblies 16a and 16b per FIG. 1 may be reduced through the use of a unitary electrode 40 having a center tubular shaft 18c holding within its lumen, the wires 32 of first electrode 22a and a second concentric tubular shaft 42 positioned about shaft 18c and holding between its walls and shaft 18c wires 44 of the second electrode 22b. Wires 44 may be tempered and formed into a shape similar to that of wires 32 described above. Shaft 18c and 42 are typically metallic and thus are coated with insulating coatings 45 and 46, respectively, to ensure that any current flow is between the exposed wires 32 rather than the shafts 18c and 42.

As mentioned above, this insulating coating 46 is also applied to the tips of the shafts 18a and 18b of the electrode assemblies 16a and 16b of FIG. 1 to likewise ensure that current does not concentrate in a short circuit between the shafts 18a and 18b but in fact flows from the wires 32 of the wires of electrodes 22a and 22b.

Other similar shaft configurations for a unitary electrode 40 may be obtained including those having side-by-side shafts 18a and 18b attached by welding or the like.

Kits of unitary electrode 40 each having different separations between first electrode 22a and second electrode 22a may be offered suitable for different tumor sizes and different tissue types.

As mentioned briefly above, in either of the embodiments of FIGS. 1 and 3 the wires 32 may include insulating coating 46 on their distal ends removed from shafts 18c and 42 so as to reduce high current densities associated with the ends of the wires 32.

In a preferred embodiment, the wires of the first and second electrodes 22a and 22b are angularly staggered (unlike as shown in FIG. 2) so that an axial view of the electrode assembly reveals equally spaced non-overlapping wires 32. Such a configuration is also desired in the embodiment of FIG. 2, although harder to maintain with two electrode assemblies 16a and 16b.

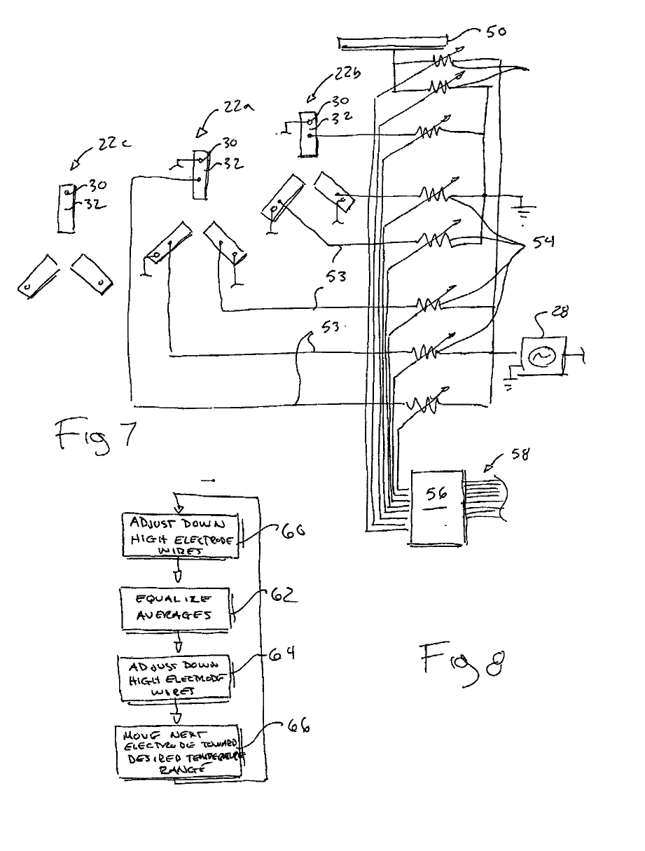

The frequency of the power oscillator 28 may be preferentially set to a value much below the 450 kHz value used in the prior art. Referring to FIG. 6, at less than 100 kHz and being most pronounced and frequencies below 10 kHz, the impedance of normal tissue increases to significantly greater than the impedance of tumor tissue. This difference in impedance is believed to be the result of differences in interstitial material between tumor and regular cell tissues although the present inventors do not wish to be bound by a particular theory. In any case, it is currently believed that the lower impedance of the tumorous tissue may be exploited to preferentially deposit energy in that tissue by setting the frequency of the power oscillator 28 at values near 10 kHz. Nevertheless, this frequency setting is not required in all embodiments of the present invention.

Importantly, although such frequencies may excite nerve tissue, such as the heart, such excitation is limited by the present bi-polar design.

Referring now to FIG. 5, the local environment of the electrodes 22a and 22b may differ by the presence of a blood vessel or the like in the vicinity of one electrode such as substantially reduces the heating of the lesion 14 in that area. Accordingly, it may be desired to increase the current density around one electrode 22a and 22b without changing the current density around the other electrode 22a and 22b. This may be accomplished by use of a skin contact plate 50 of a type used in the prior art yet employed in a different manner in the present invention. As used herein, the term contact plate 50 may refer generally to any large area conductor intended but not necessarily limited to contact over a broad area at the patient's skin.

In the embodiment of FIG. 5, the contact plate 50 may be referenced through a variable resistance 52 to either of the output of power oscillator 28 or ground per switch 53 depending on the temperature of the electrodes 22a and 22b. Generally, switch 53 will connect the free end of variable resistance 52 to the output of the power oscillator 28 when the temperature sensors 30 indicate a higher temperature on electrode 22b than electrode 22a. Conversely, switch 53 will connect the free end of variable resistance 52 to ground when the temperature sensors 30 indicate a lower temperature on electrode 22b than electrode 22a. The comparison of the temperatures of the electrodes 22a and 22b may be done via maximum determining circuits 34a and 34b, similar to that described above with respect to FIG. 2. The switch 53 may be a comparator driven solid-state switch of a type well known in the art.

The output of the maximum determining circuits 34a and 34b each connected respectively to the temperature sensors 30 of electrodes 22a and 22b may also be used to control the setting of the potentiometer 52. When the switch 53 connects the resistance 52 to the output of the power oscillator 28, the maximum determining circuits 34a and 34b serve to reduce the resistance of resistance 52 as electrode 22b gets relatively hotter. Conversely, when the switch 53 connects the resistance 52 to ground, the maximum determining circuits 34a and 34b serve to reduce the resistance of resistance 52 as electrode 22a gets relatively hotter. The action of the switch 53 and switch 52 is thus generally to try to equalize the temperature of the electrodes 22a and 22b.

If electrode 22a is close to a heat sink such as a blood vessel when electrode 22b is not, the temperature sensors 30 of electrode 22a will register a smaller value and thus the output of maximum determining circuit 34a will be lower than the output of maximum determining circuit 34b.

The resistance 52 may be implemented as a solid state devices according to techniques known in the art where the relative values of the outputs of maximum determining circuits 34a and 34b control the bias and hence resistance of a solid state device or a duty cycle modulation of a switching element or a current controlled voltage source providing the equalization described above.

Referring now to FIG. 7, these principles may be applied to a system in which each wire 32 of electrodes 22a and 22b is electrically isolated within the electrode assemblies 16a and 16b and driven by separate feeds 53 through variable resistances 54 connected either to the power oscillator 28 or its return. Electrically isolated means in this context that there is not a conductive path between the electrodes 22a and 22b except through tissue prior to connection to the power supply or control electronics. As noted before, a phase difference can also be employed between separate feeds 53 to further control the path of current flow between electrode wires 32. This phase difference could be created, e.g. by complex resistances that create a phase shift or by specialized waveform generators operating according to a computer program to produce an arbitrary switching pattern. The values of the resistances 54 are changed as will be described by a program operating on a controller 56. For this purpose, the variable resistances 54 may be implemented using solid-state devices such as MOSFET according to techniques known in the art.

Likewise, similar variable resistances 54 also controlled by a controller 56 may drive the contact plate 50.

For the purpose of control, the controller 56 may receive the inputs from the temperature sensors 30 (described above) of each wire 32 as lines 58. This separate control of the voltages on the wires 32 allows additional control of current flows throughout the tumor 12 to be responsive to heat sinking blood vessels or the like near any one wire.

Referring to FIG. 8, one possible control algorithm scans the temperature sensors 30 as shown by process block 60. For each temperature sensor 30, if the temperature at that wire 32 is above a “ceiling value” below a tissue charring point, then the voltage at that wire is reduced. This “hammering down” process is repeated until all temperatures of all wires are below the ceiling value.

Next at process block 62, the average temperature of the wires on each electrode 22a and 22b is determined and the voltage of the contact plate 50 is adjusted to incrementally equalize these average values. The voltage of the contact plate 50 is moved toward the voltage of the electrode 22 having the higher average.

Next at process block 64 the hammering down process of process block 60 is repeated to ensure that no wire has risen above its ceiling value.

Next at process block 66 one wire in sequence at each occurrence of process block 66 is examined and if its temperature is below a “floor value” below the ceiling value but sufficiently high to provide the desired power to the tumor, the voltage at that wire 32 is moved incrementally away from the voltage of the wires of the other electrode 22. Conversely, if the wire 32 is above the floor value, no action is taken.

Incrementally, each wire 32 will have its temperature adjusted to be within the floor and ceiling range by separate voltage control.

As shown in FIG. 7, this process may be extended to an arbitrary number of electrodes 22 including a third electrode set 22c whose connections are not shown for clarity.

While this present invention has been described with respect to umbrella probes, it will be understood that most of its principles can be exploited using standard needle probes energized in a bipolar configuration. Further it will be understood that the present invention is not limited to two electrode sets, but may be used with multiple electrode sets where current flow is predominantly between sets of the electrodes. The number of wires of the umbrella electrodes is likewise not limited to three and commercially available probes suitable for use with the present invention include a 10 wire version. Further although the maximum temperatures of the electrodes were used for control in the above-described examples, it will be understood that the invention is equally amenable to control strategies that use average temperature or that also evaluate minimum temperatures.

It is specifically intended that the present invention not be limited to the embodiments and illustrations contained herein, but modified forms of those embodiments including portions of the embodiments and combinations of elements of different embodiments as come within the scope of the following claims.

We claim:

1. A method of tissue ablation in a patient comprising the steps of:
(a) inserting a support shaft at a tumor volume, the support shaft having a shaft tip and shank portion adjacent to the tip, so that the shaft tip is at first locations adjacent to the tumor volume and offset from a center of the tumor volume and the shaft shank is at a second location opposed and at a predetermined separation from the first location about the tumor volume;
(b) extending first and second electrically isolated wire electrodes sets radially from the shaft and the first and second locations respectively to an extension radius; and
(c) connecting a power supply between the first and second electrode sets to induce a current flow between them through the tumor volume.

2. The method of claim 1 wherein the first and second electrodes sets are umbrella electrode sets having at least two electrode wires extending radially from the support shaft;
and wherein predetermined separation in not greater than six times the extension radius.

3. The method of claim 1 wherein the power supply provides an oscillating electrical voltage with an energy spectrum substantially concentrated in frequencies below 500 kHz.

4. The method of claim 3 wherein the oscillating electrical voltage has an energy spectrum substantially concentrated in frequencies below 100 kHz.

5. The electrode assembly of claim 1 wherein ends of the electrode wire sets distal to the support shaft are insulated.

6. The electrode assembly of claim 1 wherein an outer portion of the shaft between the first and second locations is electrically insulated.

7. A method of tumor ablation in a patient comprising the steps of:
(a) inserting a first electrode percutaneously at a tumor volume, the first electrode having a first support shaft with a first shaft tip, so that the first shaft tip is at first locations adjacent to the tumor volume and offset from a center of the tumor volume;
(b) inserting a second electrode percutaneously at the tumor volume, the second electrode having a second support shaft with a second shaft tip, so that the second support shaft is generally parallel and adjacent to the first support shaft, and so that the second shaft tip is at a second location opposed and at a predetermined separation from the first location about the tumor volume;
(c) extending first and second electrically isolated wire umbrella electrodes sets radially from the first and second shaft tips to an extension radius; and
(d) connecting a power supply between the first and second electrode umbrella sets to induce a current flow between them through the tumor volume whereby current induced heating is concentrated in the tumor volume.

8. The method of claim 7 wherein the first and second electrodes sets are umbrella electrode sets having at least two electrode wires extending radially from the support shaft;
and wherein predetermined separation in not greater than six times the extension radius.

9. The method of claim 7 wherein the power supply provides an oscillating electrical voltage with an energy spectrum substantially concentrated in frequencies below 100 kHz.

10. The method of claim 9 wherein the oscillating electrical voltage has an energy spectrum substantially concentrated in frequencies below 10 kHz.

11. The electrode assembly of claim 7 wherein ends of the electrode wire sets distal to the support shaft are insulated.

12. The electrode assembly of claim 7 wherein an outer portion of the shaft between the first and second locations is electrically insulated.

13. A method of tumor ablation in a patient comprising the steps of:
(a) inserting first and second electrically isolated electrodes percutaneously at a tumor volume, so that the first electrode is at first locations adjacent to the tumor volume and offset from a center of the tumor volume and the second electrode is at a second location opposed from the first location about the tumor volume;
(c) connecting an alternating current power supply between the first and second electrode sets to induce a current flow between them through the tumor volume, a principal frequency of the current flow being less than 100 KHz.

14. The method of claim 13 wherein principal frequency of the current flow is less than 10 kHz.

15. An electrode assembly for ablating tumors in a patient comprising:
(a) a support shaft having a shaft tip and shank portion adjacent to the tip, the shaft sized for percutaneous placement of a shaft tip adjacent at a first locations adjacent to a tumor volume and offset from a center of the tumor volume and the shaft shank at a second location opposed from the first location about the tumor volume; the shaft further having an electrically insulated outer surface between the first and second locations;
(b) first and second wire electrodes sets extensible radially from the shaft and the first and second locations respectively to an extension radius; and
(c) a power supply connected between the firs and second electrode sets to induce a current flow through the tumor volume.

16. An electrode assembly for ablating tumors in a patient comprising:
(a) a support shaft having a shaft tip and shank portion adjacent to the tip, the shaft sized for percutaneous placement of a shaft tip adjacent at a first locations adjacent to a tumor volume and offset from a center of the tumor volume and the shaft shank at a second location opposed from the first location about the tumor volume;;
(b) first and second wire electrodes sets extensible radially from the shaft and the first and second locations respectively to an extension radius, distal ends of the wire electrodes having insulating caps; and
(c) a power supply connected between the first and second electrode sets to induce a current flow through the tumor volume.

17. A method of tumor ablation in a patient comprising the steps of:
(a) inserting at least a first and second electrically isolated electrodes percutaneously at a tumor volume, so that the first electrode is at first locations adjacent to the tumor volume and offset from a center of the tumor volume and the second electrode is at a second location opposed from the first location about the tumor volume;
(b) placing a third electrically isolated electrode in electrical communication with the tumor volume; and
(c) connecting power supply between the first, second and third electrodes to independently control the current flow at the first and second electrodes.

18. The method of claim 17 further including the step of monitoring an electrode parameter at the first and second electrodes selected from the group consisting of electrode current and electrode temperature and at step (c) controlling the power supply as a function of the electrode parameters.

19. The method of claim 17 wherein the third electrode is a conductive plate against the skin of the patient.

20. The method of claim 17 wherein the third electrode is a percutaneous electrode.
